(19) United States
(12) Patent Application Publication
(10) Pub. No.: US 2026/0069233 A1
(43) Pub. Date: Mar. 12, 2026

(54) METHOD AND APPARATUS FOR DRIVING MEDICAL DEVICE, AND MEDICAL SYSTEM

(71) Applicant: GE Precision Healthcare LLC, Waukesha, WI (US)

(72) Inventors: Xueli Wang, Beijing (CN); Yigang Xia, Beijing (CN); Xiaokun Huang, Beijing (CN)

(21) Appl. No.: 19/320,461

(22) Filed: Sep. 5, 2025

(30) Foreign Application Priority Data
Sep. 6, 2024 (CN) 202411247055.2

(51) Int. Cl. A61B 6/00; A61B 6/03; G10L 15/06; G10L 15/22; G10L 17/18; G10L 17/22

(52) U.S. Cl. CPC A61B 6/54; A61B 6/032; G10L 15/063; G10L 15/22; G10L 17/18; G10L 17/22; A61B 2560/0493; G10L 2015/223

(57) ABSTRACT

Embodiments of the present application provide a method and apparatus for controlling a medical device, and a medical system. The method includes receiving a speech instruction from a sound pickup apparatus, inputting the speech instruction into a deep learning neural network, and, on the basis of the deep learning neural network, outputting an instruction for controlling the medical device, and controlling movement of the medical device according to the instruction. Therefore, by means of AI/ML-based speech control, the medical device can be accurately controlled without assistance of multiple people, which can reduce labor costs, improve efficiency, and reduce the risk of surgery failing.

### CROSS-REFERENCE TO RELATED APPLICATIONS

This application claims priority to Chinese Application No. 202411247055.2, filed on Sep. 6, 2024, the disclosure of which is incorporated herein by reference in its entirety.

### TECHNICAL FIELD

Embodiments of the present application relate to the technical field of medical devices, and relate in particular to a method and apparatus for driving a medical device, and a medical system.

### BACKGROUND

A medical device includes a medical imaging device that is configured to scan a patient or subject in a non-invasive manner, thereby acquiring a medical image of an anatomical tissue of interest of the patient to assist doctors in making a diagnosis. As an example, Computed Tomography (CT) utilizes accurately collimated X-ray beams, together with a highly sensitive detector, to perform cross-sectional scans one by one around a certain site of a human body, which is characterized by a short scanning time and image clarity, and can be used to detect a variety of diseases.

A medical imaging device such as a CT device has a driving assembly or a separate controller disposed on a device gantry, a scanning table (which may also be referred to as a patient table), or an operating table outside the scanning room. During a scan, a subject under examination is placed on the scanning table, and an operator of the medical imaging device can control the scanning table to move in translation, up or down by operating the driving assembly to complete specific medical imaging.

As an example, in a scenario in which the medical imaging device assists treatment, an assistant surgeon or imaging technician controls the medical imaging device by means of a remote controller to obtain medical diagnostic images of a patient, so as to assist a primary surgeon in performing surgery. With a puncture operation as an example, the primary surgeon needs to observe the medical diagnostic images and is responsible for executing the puncture operation, and the assistant surgeon manipulates the medical device on the basis of the instructions of the primary surgeon, to assist the primary surgeon in determining an insertion position of a needle, checking the position of the needle in real time, etc.

### SUMMARY OF THE INVENTION

The inventors have found that when driving a medical device to perform medical diagnosis and treatment, collaboration between multiple people is often necessary. For example, a primary surgeon requires assistance from an assistant surgeon who controls the medical device by manipulating a remote controller, which increases labor costs. In addition, if the assistant surgeon cannot promptly understand the instructions from the primary surgeon, surgery time can easily become extended and operational errors could even occur, thereby leading surgery to fail. Therefore, how to reduce labor costs, improve efficiency, and reduce the risk of surgery failing is a problem that needs to be solved.

In view of at least one of the above technical problems or other similar problems, embodiments of the present application provide a method and apparatus for driving a medical device, and a medical system.

According to an aspect of the embodiments of the present application, a method for driving a medical device is provided, including receiving a speech instruction from a sound pickup apparatus, inputting the speech instruction into a deep learning neural network, and, on the basis of the deep learning neural network, outputting a driving instruction for driving the medical device, and driving movement of the medical device according to the driving instruction.

In some embodiments, on the basis of the deep learning neural network, outputting the driving instruction for driving the medical device includes performing automatic speech recognition (ASR) on the speech instruction on the basis of a first deep learning neural network to perform feature extraction and output a system-recognized speech word, and performing natural language processing (NLP) on the system-recognized speech word on the basis of a second deep learning neural network to perform semantic analysis and output the driving instruction for driving the medical device.

In some embodiments, the method further includes selecting a corresponding driving instruction for the speech instruction according to pre-stored custom information; wherein the custom information comprises a correspondence between speech instructions and driving instructions.

In some embodiments, the method further includes performing voiceprint detection on the speech instruction on the basis of pre-stored voiceprint information, and outputting the system-recognized speech word when the speech instruction matches a voiceprint feature of an authorized user; and not outputting the system-recognized speech word when the speech instruction does not match the voiceprint feature of the authorized user.

In some embodiments, the method further includes performing on/off detection on the speech instruction on the basis of pre-stored wake-up information/shut-down information when the speech instruction matches the voiceprint feature of the authorized user, and enabling driving of the medical device when the speech instruction comprises the wake-up information; and disabling the driving of the medical device when the speech instruction comprises the shut-down information.

In some embodiments, the method further includes training the deep learning neural network by using a training sample.

In some embodiments, training the deep learning neural network by using the training sample includes performing speech command recognition (SCR) on the training sample on the basis of the deep learning neural network to perform feature extraction and output a feature vector, determining a difference between the feature vector and a feature vector of another speech instruction according to a semantic distance, and determining the training sample as a valid sample when the difference is greater than or equal to a preset threshold.

In some embodiments, the method further includes selecting a corresponding driving instruction for the training sample and storing custom information, wherein the custom information comprises a correspondence between speech instructions and driving instructions.

According to another aspect of the embodiments of the present application, an apparatus for driving a medical device is provided, comprising a processor and a memory, wherein the processor is configured as the foregoing method for driving the medical device.

According to still another aspect of the embodiments of the present application, a medical system is provided, including a sound pickup apparatus, which receives a speech instruction from a user, a driving apparatus, which inputs the speech instruction into a deep learning neural network and, on the basis of the deep learning neural network, outputs a driving instruction for driving a medical device, and a medical device, which performs movement according to the driving instruction.

In some embodiments, the medical system further includes a display device, which displays an image acquired by the medical device and the speech instruction recognized by the driving apparatus. In some embodiments, the display device further displays historical information of speech instructions from the user within a period of time. In some embodiments, the sound pickup apparatus comprises a wearable microphone fixed to the user or a microphone fixed to the medical device.

One of the beneficial effects of the embodiments of the present application is that: a speech instruction is input into a deep learning neural network, and a driving instruction for driving a medical device is output on the basis of the deep learning neural network. Therefore, by means of AI/ML-based speech control, the medical device can be accurately driven without assistance of multiple people, which can reduce labor cost, improve efficiency, and reduce the risk of surgical failure. Moreover, compared with a conventional driving assembly or controller, in the technical solutions of the present application, doctors do not need to manually operate a driving assembly or controller, and an operating room or a scanning room may not be provided with a driving assembly or controller. Correspondingly, spatial obstruction faced by the doctors in the operating room or the scanning room is less, that is, the degree of freedom for the doctors to move or operate in the operating room or the scanning room is higher.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a CT device according to an embodiment of the present application;

FIG. 2 is a schematic diagram of a CT imaging system according to an embodiment of the present application;

FIG. 3 is a schematic diagram of a CT device and a driving apparatus thereof according to an embodiment of the present application;

FIG. 4 is a schematic diagram of a method for driving a medical device according to an embodiment of the present application;

FIG. 5 is a schematic diagram on the basis of a deep learning neural network according to an embodiment of the present application;

FIG. 6 is an example diagram of generating a driving instruction on the basis of a speech instruction according to an embodiment of the present application;

FIG. 7 is an example diagram of a custom correspondence according to an embodiment of the present application;

FIG. 8 is another schematic diagram of a method for driving a medical device according to an embodiment of the present application;

FIG. 9 is a schematic diagram of deep learning neural network training according to an embodiment of the present application;

FIG. 10 is an example diagram of deep learning neural network training according to an embodiment of the present application;

FIG. 11 is a schematic diagram of confirmation during training according to an embodiment of the present application;

FIG. 12 is a schematic diagram of a medical system according to an embodiment of the present application; and

FIG. 13 is an example diagram of a display interface of a display device according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than being limited to the meaning of “one”. In addition, the term “the” should be construed as including both the singular and plural forms, unless otherwise specified in the context. In addition, the term “according to” should be construed as “at least partially according to. ” and the term “based on” should be construed as “at least partially based on. ”, unless otherwise explicitly specified in the context.

The features described and/or illustrated for one implementation may be used in one or more other implementations in the same or similar way, be combined with features in other implementations, or replace features in other implementations. The terms “include/comprise” when used herein refer to the presence of features, integrated components, steps, or assemblies, but do not preclude the presence or addition of one or more other features, integrated components, steps, or assemblies.

The medical device described in the present application includes, for example, a medical imaging device. The present application is not limited thereto, and may be applied to any medical device that can be driven to perform various movements. The medical imaging device (e.g., a CT device) is taken as an example for description below.

The medical imaging device is applicable to various medical imaging modalities, and includes, but is not limited to, Computed Tomography (CT) imaging devices, or Positron Emission Tomography (PET)-CT, Magnetic Resonance Imaging (MRI), or any other suitable medical imaging devices.

The system obtaining the medical imaging data may include the aforementioned medical imaging device, and may include a separate computer device connected to the medical imaging device, and may further include a computer device connected to an Internet cloud, the computer device being connected by means of the Internet to the medical imaging device or a memory for storing medical images. The imaging method may be independently or jointly implemented by the aforementioned medical imaging device, the computer device connected to the medical imaging device, and the computer device connected to the Internet cloud. For example, the system obtaining the medical image data may be a CT imaging system, etc.

As an example, the embodiments of the present application are described below in conjunction with an X-ray computed tomography (CT) imaging device. Those skilled in the art would appreciate that the embodiments of the present application can also be applied to other medical devices.

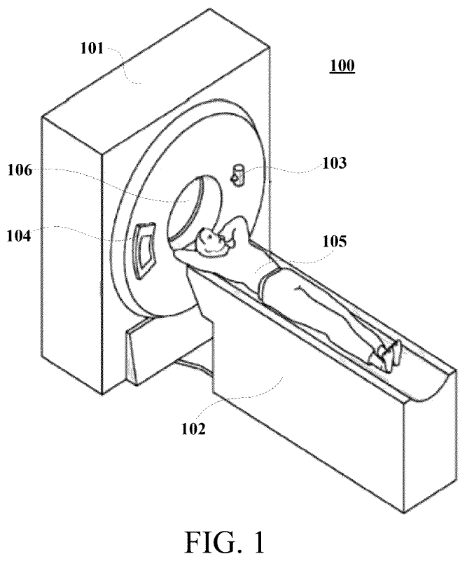

FIG. 1 is a schematic diagram of a CT device according to an embodiment of the present application, and schematically shows a CT device 100. As shown in FIG. 1, the CT device 100 includes a scanning gantry 101 and a patient table 102 (for example, a scanning table). The scanning gantry 101 has an X-ray source 103, and the X-ray source 103 projects an X-ray beam toward a detector assembly or collimator 104 on an opposite side of the scanning gantry 101. A subject under examination 105 can lie flat on the patient table 102 and be moved into a scanning gantry opening 106 along with the patient table 102. Medical image data of the subject under examination 105 can be obtained by means of scanning performed by the X-ray source 103.

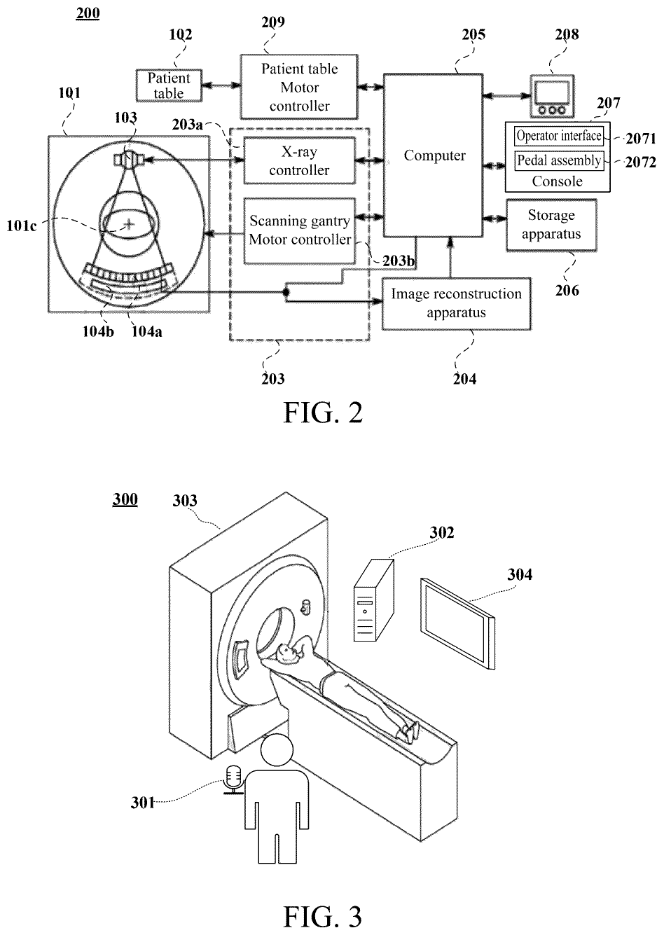

FIG. 2 is a schematic diagram of a CT imaging system according to an embodiment of the present application, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the subject under examination 105.

The DAS 104b, according to the sensing of the detector units 104a, converts collected information into projection data for subsequent processing. During the scanning for acquiring the X-ray projection data, the scanning gantry 101 and components mounted thereon rotate around a center of rotation 101c.

The rotation of the scanning gantry 101 and the operation of the X-ray source 103 are controlled by a control mechanism 203 of the CT imaging system 200. The control mechanism 203 includes an X-ray controller 203a that provides power and a timing signal to the X-ray source 103 and a scanning gantry motor controller 203b that controls the rotational speed and position of the scanning gantry 101. An image reconstruction apparatus 204 receives the projection data from the DAS 104b and executes image reconstruction. A reconstructed image is transmitted as an input to a computer 205, and the computer 205 stores the image in a mass storage apparatus 206.

The computer 205 also receives commands and scanning parameters from an operator by means of a console 207. The console 207 has an operator interface 2071 in a certain form, such as a keyboard, a mouse, or a speech activated controller. The console 207 may also have an input apparatus such as a pedal assembly 2072. In addition, the console 207 may have another suitable input apparatus. An associated display 208 allows the operator to observe a reconstructed image and other data from the computer 205. The commands and parameters provided by the operator are used by the computer 205 to provide control signals and information to the DAS 104b, the X-ray controller 203a, and the scanning gantry motor controller 203b. Additionally, the computer 205 operates a patient table motor controller 209 which controls the patient table 102 so as to position the subject under examination 105 and the scanning gantry 101. In particular, the patient table 102 moves the subject under examination 105 to, fully or in part, pass through the scanning gantry opening 106 in FIG. 1.

The device and system for acquiring medical image data (which may also be referred to as medical images or medical image data) according to the embodiments of the present application are schematically described above, but the present application is not limited thereto. The medical imaging device may be a CT device, a PET-CT, or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS)”. SaaS can exist between hospitals, between a hospital and an imaging center, or between a hospital and a third-party online diagnosis and treatment service provider.

Medical image scanning is schematically illustrated above, and the embodiments of the present application are described in detail below with reference to the drawings. In the embodiments described below, the medical device being a CT device is taken as an example for description, and the content of the description is also applicable to other medical devices.

FIG. 3 is a schematic diagram of a CT device and a driving device thereof according to an embodiment of the present application, and schematically shows a block diagram of the driving device thereof by taking the CT device as an example. As shown in FIG. 3, a medical system 300 includes, for example, a sound pickup apparatus 301 (e.g., a microphone), a driving apparatus (e.g., a controller, a processing device, such as a computer including a processor) 302 for a CT device 303, the CT device 303, and a display device 304. The driving apparatus 302 for the CT device 303 converts a received speech instruction into a driving instruction (e.g., instructions executable by the processor), so as to control the CT device 303 to perform an action and control the display device 304 to display.

For example, when a user issues a speech instruction, the sound pickup apparatus 301 sends the speech instruction to the driving apparatus 302 for the CT device 303. The driving apparatus 302 for the CT device 303 processes the speech instruction and outputs a driving instruction. The CT device 303 performs a corresponding action according to the driving instruction, and the display device 304 may also perform corresponding display according to the speech instruction.

The sound pickup apparatus 301 includes a wearable microphone fixed to the user or a microphone fixed to the medical device. For example, the sound pickup apparatus 301 may be located on a user side, such as in a wearable microphone device, or the sound pickup apparatus 301 may be integrated in the medical device 303, or the sound pickup apparatus 301 may also exist independently at other locations (for example, a sound receiving device hung on a support).

The driving apparatus 302 for the medical device 303 may include a processor, a memory, and a driving module (driving program). For example, the driving module may be located in the medical device 303 in the form of software, or located in a server outside the medical device 303, or independently exist in a remote cloud system. The display device 304 may exist independently (e.g., as shown in FIG. 3), or may be combined with the medical device.

The above schematically describes some constituent structures of the embodiments of the present application, and the present application is not limited thereto. The driving method for the embodiments of the present application will be schematically described below.

The embodiments of the present application provide a method for driving a medical device, which drives the medical device on the basis of a speech instruction of a user.

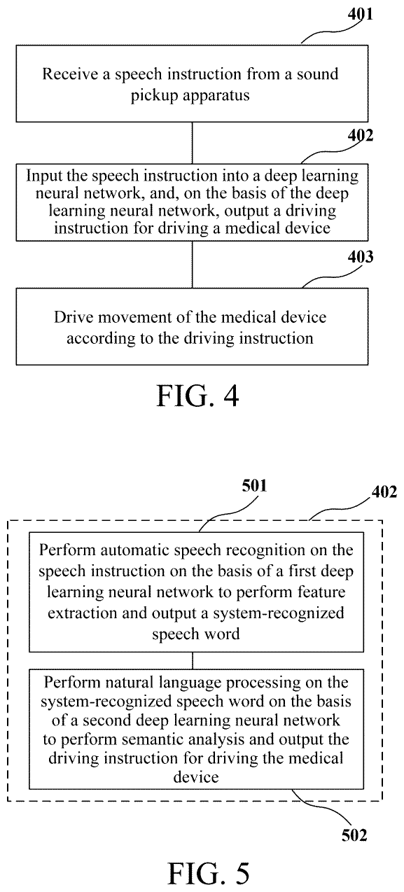

FIG. 4 is a schematic diagram of a method for driving a medical device according to an embodiment of the present application, which is described from the side of a driving apparatus for the medical device. As shown in FIG. 4, the method includes 401, receiving a speech instruction from a sound pickup apparatus; 402, inputting the speech instruction into a deep learning neural network, and, on the basis of the deep learning neural network, outputting a driving instruction for driving the medical device; and 403, driving movement of the medical device according to the driving instruction.

It is worth noting that FIG. 4 merely schematically describes the embodiments of the present application, but the present application is not limited thereto. For example, some of the above steps may be executed simultaneously, or may be executed in a sequential order. The order of execution between operations may be appropriately adjusted. In addition, some other operations may be added or some operations may be omitted. Those skilled in the art may make appropriate variations according to the above content, rather than being limited to the above disclosure of FIG. 4.

In the embodiments of the present application, the speech instruction of the user is acquired by means of the sound pickup apparatus, the speech instruction is input into the deep learning neural network, and the driving instruction for driving the medical device is output on the basis of the deep learning neural network. Therefore, by means of Artificial Intelligence/Machine Learning (AI/ML)-based speech control, the medical device can be accurately driven without assistance of multiple people, which can reduce labor cost, improve efficiency, and reduce the risk of surgical failure.

In addition, compared with a conventional driving assembly or controller, in the technical solutions of the present application, doctors do not need to manually operate a driving assembly or controller, and an operating room or a scanning room may not be provided with a driving assembly or controller. Correspondingly, spatial obstruction faced by the doctors in the operating room or the scanning room is less, that is, the degree of freedom for the doctors to move or operate in the operating room or the scanning room is higher.

In some embodiments, the sound pickup apparatus may be any form of sound receiving apparatus, such as a headset, a microphone clipped on the user's clothes, an independent sound receiving device, or a sound receiving module fixed on the medical device, and the embodiments of the present application are not limited thereto.

In some embodiments, the speech instruction may be an instruction preset by the driving apparatus for the medical device at the factory, or may be a valid instruction customized by an authorized user. The language of the speech instruction may be Chinese, English, Japanese, Korean, or the like, or may be standard Mandarin or a dialect, and the embodiments of the present application are not limited thereto.

For example, the speech instruction may indicate the direction and/or magnitude of movement of the medical device, such as “move forward by one space”, “move downward”, “downward”, “upward”, “move leftward by 2 centimeters”, etc. For another example, the speech instruction may indicate a state of the medical device, such as “ON”, “OFF”, etc. For still another example, the speech instruction may indicate a function of the medical device, such as “activate a speech control function”, “deactivate a speech control function”, “increase illumination brightness”, etc. For yet another example, the speech instruction may indicate a state of a user, such as “activate user B”, “deactivate user C”, etc.

In some embodiments, the deep learning neural network may use an existing open source AI/ML model, which may be selected according to the needs of accuracy during specific implementation, which is not limited in the present application. For the specific content of the deep learning neural network, reference can be made to the related art.

FIG. 5 is a schematic diagram on the basis of a deep learning neural network according to an embodiment of the present application. As shown in FIG. 5, on the basis of the deep learning neural network, outputting the driving instruction for driving the medical device may include 501, performing Automatic Speech Recognition (ASR) on the speech instruction on the basis of a first deep learning neural network to perform feature extraction and output a system-recognized speech word; and 502, performing Natural Language Processing (NLP) on the system-recognized speech word on the basis of a second deep learning neural network to perform semantic analysis and output the driving instruction for driving the medical device.

Therefore, the accuracy of speech recognition can be further improved by combining two AI/ML modules. For example, the speech of different people in different regions can be adapted to, and even non-standard speech or a dialect can be accurately recognized, thereby improving the robustness and scalability of speech recognition.

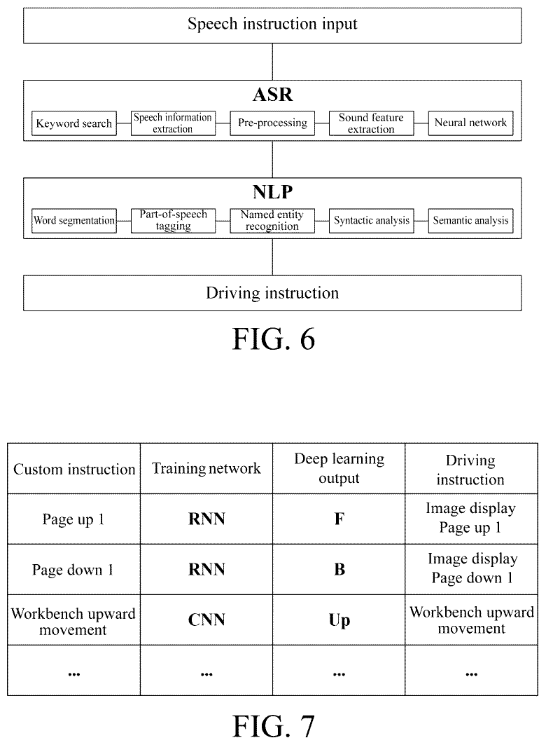

FIG. 6 is an example diagram of generating a driving instruction on the basis of a speech instruction according to an embodiment of the present application.

As shown in FIG. 6, for example, ASR may include: keyword search, speech information extraction, speech information preprocessing, sound feature extraction, neural network processing, etc., but is not limited thereto. Specifically, for example, a keyword in the input speech instruction is determined on the basis of the speech instruction predefined by the factory and the speech instruction set by the authorized user; the speech instruction issued by the user is recognized and extracted on the basis of the keyword to remove background noise contained in the speech instruction; and preprocessing and feature extraction operations are performed on the extracted speech instruction.

For example, the preprocessing operation includes, but is not limited to, noise reduction, resampling, and channel coordination, and the feature extraction operation includes, but is not limited to, short-time Fourier transform, Mel-frequency cepstral coefficients, linear predictive coding, and cepstral coefficients based on perceptual features; and the present application is not limited thereto.

As shown in FIG. 6, the extracted speech features may be input into a deep learning neural network to output a system-recognized speech word. The deep learning neural network includes, but is not limited to, a recurrent neural network, a long short-term memory network, a convolutional neural network, or a transformer network.

As shown in FIG. 6, NLP may include: word segmentation, part-of-speech tagging, named entity recognition, syntactic analysis, semantic analysis, etc., but is not limited thereto. Specifically, for example, a rule for word segmentation is determined on the basis of a language corresponding to the speech instruction, and a word segmentation operation is performed on the speech word output by the neural network according to the rule; part-of-speech tagging and named entity recognition operations are performed on each word to assist in the implementation of syntactic analysis; a hierarchical structure of a sentence and a dependency relationship between words are determined using syntactic analysis on the basis of grammatical rules of the language; and a machine is assisted by means of semantic analysis to recognize the speech word.

Therefore, combining ASR and NLP can further improve the accuracy of speech recognition.

In some embodiments, a corresponding driving instruction may be selected for the speech instruction according to pre-stored custom information; and the custom information includes a correspondence between speech instructions and driving instructions.

FIG. 7 is an example diagram of a customized correspondence according to an embodiment of the present application, and the correspondence may be pre-stored. A corresponding driving instruction may be selected for the speech instruction according to pre-stored custom information.

For example, the speech instruction is input into a neural network, and an output result may be obtained; and on the basis of the output result and according to the custom relationship, a corresponding driving instruction may be matched, thereby achieving the correspondence between the speech instruction and the driving instruction. As shown in FIG. 7, for example, the same neural network or different neural networks may be used for different speech instructions, and the embodiments of the present application are not limited thereto.

Therefore, a quick correspondence between the speech instruction and the driving instruction may be achieved, thereby improving the response speed of the medical device. In addition, instructions and operations can be conveniently and flexibly bound by using the custom information, thereby further improving the robustness and scalability of speech recognition.

In some embodiments, voiceprint detection may be performed on the speech instruction on the basis of pre-stored voiceprint information; when the speech instruction matches a voiceprint feature of an authorized user, a system-recognized speech word is output; and when the speech instruction does not match the voiceprint feature of the authorized user, the system-recognized speech word is not output.

For example, during the process of use by the authorized user, voiceprint detection is performed on a received speech instruction on the basis of the pre-stored voiceprint information to determine whether the speech instruction is from the authorized user. If no voiceprint feature matching the speech instruction is found in the pre-stored voiceprint information, the speech instruction is considered to come from an unauthorized user, and the first deep neural network does not output the system-recognized speech word.

Taking a surgery as an example, for example, if a primary surgeon is an authorized user, and other persons such as an assistant surgeon and a nurse are unauthorized users, only the sound of the primary surgeon can be recognized and then output as a system-recognized speech word to generate a driving instruction, and the sound of other persons cannot generate driving instructions. Even if another person issues a legal speech instruction (for example, “move left”), the medical device does not perform a corresponding action. Therefore, an unauthorized user can be prevented from driving movement of the medical device, which can improve anti-interference performance.

For another example, still taking a surgery as an example, if a voiceprint feature from authorized user A is the first voiceprint feature belonging to authorized users and detected by the medical system, during the surgery, only the voiceprint of authorized user A is recognized, and other authorized users cannot use the speech-driven function in the surgery, thereby preventing the problem of speech instructions from multiple people being confused.

For still another example, if a user who can use the speech-driven function needs to be added in the current surgery, authorized user A may issue a corresponding speech instruction, such as “activate user B”; and then, when authorized user B has input his/her voiceprint feature, the medical system can recognize the voiceprint feature of the authorized user B, that is, the authorized user B can also use the function speech-driven function in the current surgery. As an example, at this time, both authorized user A and authorized user B may perform speech driving. As another example, at this time, authorized user A is automatically deactivated and replaced by authorized user B to perform speech driving. Therefore, the flexibility of speech instruction recognition can be further improved.

In some embodiments, when the speech instruction matches the voiceprint feature of the authorized user, on/off detection is performed on the speech instruction on the basis of the pre-stored wake-up information/shut-down information; when the speech instruction includes the wake-up information, driving of the medical device is enabled; and when the speech instruction includes the shut-down information, the driving of the medical device is disabled.

For example, the authorized user may customize a “wake-up word” or use a “wake-up word” set by the factory to enable the speech-driven function. When the authorized user issues a wake-up instruction, the driving apparatus for the medical device receives the wake-up instruction from the sound pickup apparatus and processes the wake-up instruction.

For another example, whether the wake-up instruction is from an authorized user may be verified on the basis of a voiceprint comparison technology, and if so, a result is output by means of the neural network and a corresponding driving instruction is determined; and a speech assistant is waken up on the basis of the driving instruction to receive a subsequent speech instruction. The authorized user may also customize a “shut-down word” or use a “shut-down word” set by the factory to disable the speech-driven function, and the embodiments of the present application are not limited thereto. Therefore, the accuracy and safety of speech instruction recognition can be further improved, thereby improving the anti-interference performance.

The embodiments of the present application have been schematically illustrated above, but are not limited thereto. In addition, each of the above embodiments may be implemented individually, or two or more of the embodiments may be combined. For example, various embodiments may be combined when a medical device is driven by speech.

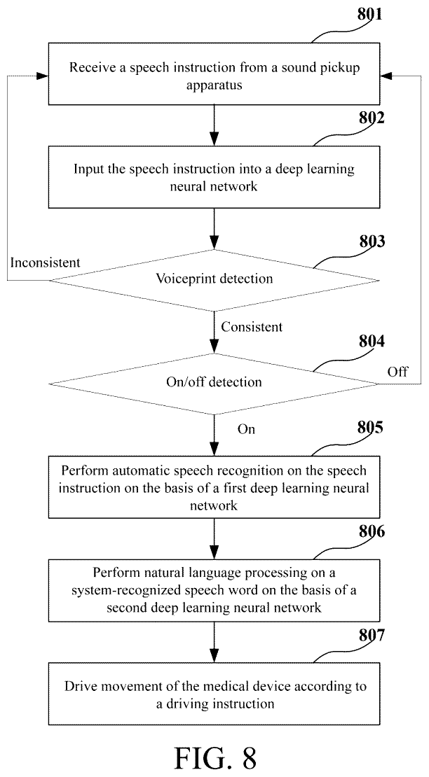

FIG. 8 is another schematic diagram of a method for driving a medical device according to an embodiment of the present application, which is described from the side of an apparatus for driving the medical device. As shown in FIG. 8, the method includes 801, receiving a speech instruction from a sound pickup apparatus; 802, inputting the speech instruction into a deep learning neural network; 803, performing voiceprint detection on the speech instruction on the basis of pre-stored voiceprint information; and executing 804 when the speech instruction matches a voiceprint feature of an authorized user, and executing 801 when the speech instruction does not match the voiceprint feature of the authorized user; 804, performing on/off detection on the speech instruction on the basis of pre-stored wake-up information/shut-down information when the speech instruction matches the voiceprint feature of the authorized user; and when the speech instruction includes the wake-up information, executing 805 to enable driving of the medical device; when the speech instruction includes the shut-down information, executing 801 to disable the driving of the medical device; 805, performing automatic speech recognition (ASR) on the speech instruction on the basis of a first deep learning neural network to perform feature extraction and output a system-recognized speech word; 806, performing natural language processing (NLP) on the system-recognized speech word on the basis of a second deep learning neural network to perform semantic analysis and output a driving instruction for driving the medical device; and 807, driving movement of the medical device according to the driving instruction.

It is worth noting that FIG. 8 merely schematically illustrates the embodiment of the present application, but the present application is not limited thereto. For example, some of the above steps may be executed simultaneously, or may be executed in a sequential order. The order of execution between operations may be appropriately adjusted (for example, the order of 803 and 804 may be interchanged). In addition, some other operations may be added or some operations may be omitted. Those skilled in the art may make appropriate variations according to the above content, rather than being limited to the above disclosure of FIG. 8.

The driving of the embodiments of the present application is schematically illustrated above, and the training of the present application will be schematically illustrated below. In some embodiments, the deep learning neural network may be trained by using a training sample. For example, offline training may be performed, for example, the deep learning neural network is trained before speech recognition, and then the trained deep learning neural network is used for driving an actual medical device. For another example, online training may also be performed, for example, a model is trained simultaneously during the driving process of the actual medical device. For still another example, offline training and online training may be combined. Therefore, the accuracy and reliability of speech recognition can be further improved by means of model training.

In some embodiments, training the deep learning neural network by using the training sample includes: performing speech command recognition (SCR) on the training sample on the basis of the deep learning neural network to perform feature extraction and output a feature vector; determining a difference between the feature vector and a feature vector of another speech instruction according to a semantic distance; and when the difference is greater than or equal to a preset threshold, determining the training sample as a valid sample.

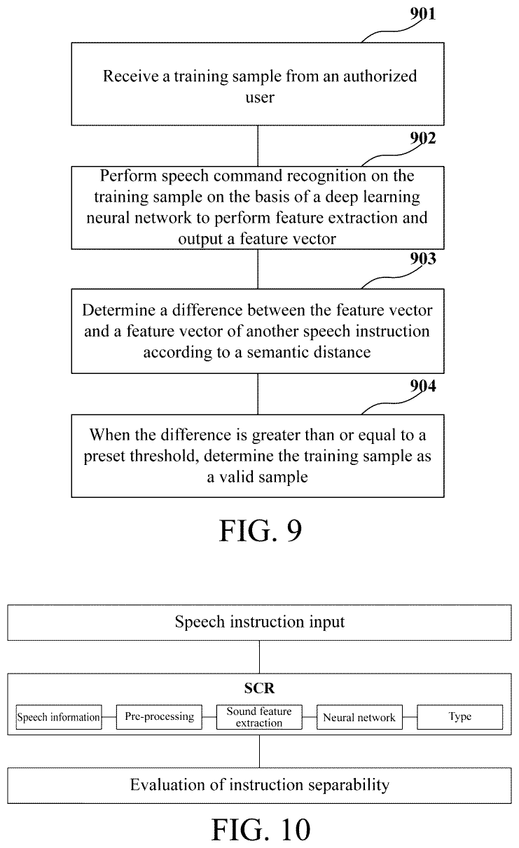

FIG. 9 is a schematic diagram of deep learning neural network training according to an embodiment of the present application. As shown in FIG. 9, the method includes 901, receiving a training sample from an authorized user; 902, performing speech command recognition (SCR) on the training sample on the basis of the deep learning neural network to perform feature extraction and output a feature vector; 903, determining a difference between the feature vector and a feature vector of another speech instruction according to a semantic distance; and 904, when the difference is greater than or equal to a preset threshold, determining the training sample as a valid sample.

It is worth noting that FIG. 9 merely schematically illustrates the embodiment of the present application, but the present application is not limited thereto. For example, some of the above steps may be executed simultaneously, or may be executed in a sequential order. The order of execution between operations may be appropriately adjusted. In addition, some other operations may be added or some operations may be omitted. Those skilled in the art may make appropriate variations according to the above content, rather than being limited to the above disclosure of FIG. 9.

FIG. 10 is an example diagram of deep learning neural network training according to an embodiment of the present application. As shown in FIG. 10, for example, SCR includes performing preprocessing, feature extraction, deep learning neural network training, etc., on the training sample. The preprocessing operation includes, but is not limited to, noise reduction, resampling, and channel coordination. The feature extraction operation includes, but is not limited to, short-time Fourier transform, Mel frequency cepstral coefficients, linear predictive coding, and cepstral coefficients based on perceptual features. The deep learning neural network includes, but is not limited to, a recurrent neural network, a long short-term memory network, a convolutional neural network, or a transformer network.

For example, semantic distance determination includes, but is not limited to: determining a difference between a feature vector and a feature vector of another speech instruction on the basis of a minimum decoding distance of a built-in tag. For example, currently, a speech instruction “move left” has been trained and pre-stored; and if a user says “move to the left” during training, and a difference between a feature vector of “move to the left” and a feature vector of “move left” is, for example, less than a threshold, the training sample of “move to the left” may be considered invalid.

For another example, currently, a speech instruction “move left” has been trained and pre-stored; and if the user says “move to the left by 2 centimeters” during training, and a difference between a feature vector of “move to the left by 2 centimeters” and a feature vector of “move left” is, for example, greater than a threshold, the training sample of “move to the left by 2 centimeters” may be considered valid.

Therefore, evaluation of instruction separability may be performed on the basis of the semantic distance determination, and the difference between the custom instruction and all the set instructions may be recognized, thereby determining whether the current custom instruction is concise and unambiguous, clarifying the driving instruction corresponding to the speech instruction, and preventing the execution of incorrect instructions caused by ambiguity and instruction confusion.

In some embodiments, the training method for the deep learning neural network further includes creating a correspondence table between custom speech instructions and driving instructions. For example, as shown in FIG. 7, the output result of the deep learning network is determined on the basis of the custom speech instruction of the authorized user and the current deep learning neural network, thereby determining the driving instruction corresponding to the output result.

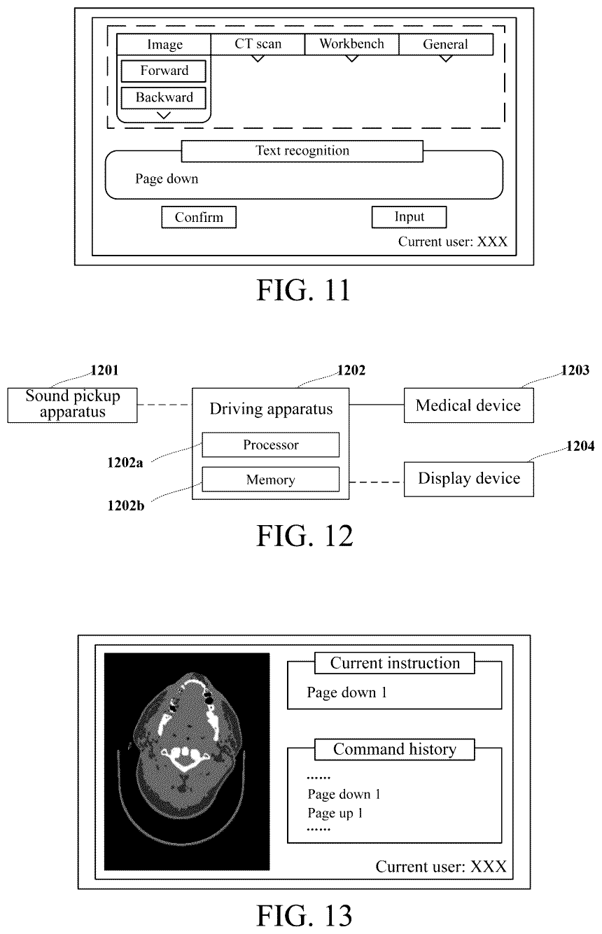

FIG. 11 is a schematic diagram of confirmation during model training according to an embodiment of the present application.

For example, as shown in FIG. 11, the authorized user selects a function he/she wants to set an instruction for, and inputs a speech instruction he/she wants to set, and a current deep learning network output result is output by means of speech command recognition. After the semantic distance determination, whether the speech instruction is a valid sample is determined; if the speech instruction is a valid sample, the speech instruction corresponds to the current function, that is, the custom speech instruction corresponds to the current driving instruction; and if the speech instruction is an invalid sample, the authorized user is reminded to reset.

Therefore, the correspondence between the speech instruction and the driving instruction can be clarified. When the medical system is used, the driving instruction corresponding to the input speech instruction is quickly determined, thereby increasing the response speed of the medical device.

The above schematically illustrates training, and the present application is not limited thereto. For specific content such as model training and semantic distance determination, reference may also be made to the related art. By learning usage habits of a certain group (e.g., authorized users within the same department), the deep learning neural network adapts to the department. Further, the deep learning neural network may also be applicable to a certain region or area, for example, by accessing a speech library of a hospital or area to obtain sufficient training samples, so as to adapt to the regional accent, the dedicated vocabulary and grammar of the hospital.

The embodiments of the present application further provide an apparatus for driving a medical device, including a processor and a memory, wherein the processor is configured to execute the foregoing method for driving the medical device. For example, the processor is configured to execute the following operations: receiving a speech instruction from a sound pickup apparatus; inputting the speech instruction into a deep learning neural network, and, on the basis of the deep learning neural network, outputting a driving instruction for driving the medical device; and driving movement of the medical device according to the driving instruction. The embodiments of the present application further provide a medical system.

FIG. 12 is a schematic diagram of a medical system according to an embodiment of the present application. The medical system includes: a sound pickup apparatus 1201, a medical device 1203, and a driving apparatus 1202 for the medical device 1203. In addition, as shown in FIG. 12, the medical system may further include a display device 1204, etc.

The sound pickup apparatus 1201 receives a speech instruction from a user; the driving apparatus 1202 inputs the speech instruction into a deep learning neural network, and, on the basis of the deep learning neural network, outputs a driving instruction for driving the medical device 1203; and the medical device 1203 performs an action according to the driving instruction. The display device 1204 displays an image acquired by the medical device and/or a speech instruction recognized by the driving apparatus 1202.

In some embodiments, the sound pickup apparatus 1201 may be any form of sound receiving apparatus, such as a wearable microphone fixed to the user or a microphone fixed to the medical device, and the embodiments of the present application are not limited thereto. The sound pickup apparatus 1201 may acquire a speech instruction of the user in real time, and is connected to the driving apparatus 1202 for the medical device in a wireless or wired manner. The driving apparatus 1202 for the medical device processes the speech information from the sound pickup apparatus 101 and outputs the driving instruction for driving the medical device on the basis of the neural network.

As shown in FIG. 12, the driving apparatus 1202 for the medical device may include: one or more processors (for example, central processing units (CPUs)) 1202a and one or more memories 1202b. The memory 1202b is coupled to the processor 1202a. The memory 1202b may store various data such as a custom instruction of an authorized user, voiceprint data, and historical information of speech instructions from the authorized user. The medical device 1203 and the display device 1204 perform actions on the basis of the received driving instruction. The medical device 1203 and the display device 1204 are connected to the driving apparatus 1202 for the medical device in a wireless or wired manner.

In some embodiments, the display device 1204 further displays historical information of speech instructions from the user within a period of time. For example, the display device 1204 not only allows the user to observe images from the CT device 1203, but also displays speech instructions recognized by the driving apparatus 1202 for the CT device and historical information of speech instructions from authorized users within a period of time.

FIG. 13 is an example diagram of a display interface of a display device according to an embodiment of the present application. For example, as illustrated in FIG. 13, the display device 1204 displays an image from the CT device 1203, a speech instruction that is currently input, and historical information (command history) of the speech instruction. For example, on the basis of a speech instruction of “Page down 1” currently input by the user, the driving apparatus 1202 controls the display device 1204 to switch the image to the next page.

Therefore, by displaying the historical information of the speech instruction, the user or other persons can confirm the speech instruction, and then can predict the action of the medical device, so that the corresponding information can be obtained by means of the display device even when the speech instruction of the authorized user is not clearly heard.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

For simplicity, the figures only exemplarily illustrate the connection relationship or signal direction between various components or modules, but it should be clear to those skilled in the art that various related technologies such as bus connection can be used. The various components or modules described above can be implemented by means of hardware such as a processor or a memory, etc. The embodiments of the present application are not limited thereto.

The embodiments of the present application further provide a computer-readable program or program product, wherein when the program is executed in an electronic device, the program causes a computer to execute, in the electronic device, the method for driving the medical device as described in the foregoing embodiments.

The embodiments of the present application further provide a storage medium having a computer-readable program stored thereon, wherein the computer-readable program causes a computer to execute, in an electronic device, the method for driving the medical device as described in the foregoing embodiments.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. A storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for executing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A method for controlling a medical device, comprising:
receiving a speech instruction from a sound pickup apparatus;
inputting the speech instruction into a deep learning neural network, and, on the basis of the deep learning neural network, outputting an instruction for controlling the medical device; and
controlling movement of the medical device according to the instruction.

2. The method according to claim 1, wherein, on the basis of the deep learning neural network, outputting the instruction for controlling the medical device comprises:
performing automatic speech recognition on the speech instruction on the basis of a first deep learning neural network to perform feature extraction and output a system-recognized speech word; and
performing natural language processing on the system-recognized speech word on the basis of a second deep learning neural network to perform semantic analysis and output the instruction for controlling the medical device.

3. The method according to claim 2, further comprising:
selecting a corresponding instruction for the speech instruction according to pre-stored custom information; wherein the custom information comprises a correspondence between speech instructions and instructions for controlling the medical device.

4. The method according to claim 2, further comprising:
performing voiceprint detection on the speech instruction on the basis of pre-stored voiceprint information; and
outputting the system-recognized speech word when the speech instruction matches a voiceprint feature of an authorized user; and not outputting the system-recognized speech word when the speech instruction does not match the voiceprint feature of the authorized user.

5. The method according to claim 4, further comprising:
performing on/off detection on the speech instruction on the basis of pre-stored wake-up information/shut-down information when the speech instruction matches the voiceprint feature of the authorized user; and
enabling driving of the medical device when the speech instruction comprises the wake-up information; and disabling driving of the medical device when the speech instruction comprises the shut-down information.

6. The method according to claim 1, further comprising:
training the deep learning neural network by using a training sample.

7. The method according to claim 6, wherein training the deep learning neural network by using a training sample comprises:
performing speech command recognition on the training sample on the basis of the deep learning neural network to perform feature extraction and output a feature vector;
determining a difference between the feature vector and a feature vector of another speech instruction according to a semantic distance; and
determining the training sample as a valid sample when the difference is greater than or equal to a preset threshold.

8. The method according to claim 6, further comprising:
selecting a corresponding instruction for the training sample and storing custom information, wherein the custom information comprises a correspondence between speech instructions and instructions for controlling the medical device.

9. A medical system, comprising:
a sound pickup apparatus, which receives a speech instruction from a user;
a controller, which inputs the speech instruction into a deep learning neural network and, on the basis of the deep learning neural network, outputs an instruction for controlling a medical device; and
the medical device, which performs movement according to the instruction.

10. The medical system according to claim 9, further comprising:
a display device, which displays an image acquired by the medical device and the speech instruction recognized by the controller.

11. The medical system according to claim 10, wherein the display device further displays historical information of speech instructions from the user within a period of time.

12. The medical system according to claim 9, wherein the sound pickup apparatus comprises a wearable microphone fixed to the user or a microphone fixed to the medical device.
